(19) United States
(12) Patent Application Publication
(10) Pub. No.: US 2024/0130948 A1
(43) Pub. Date: Apr. 25, 2024

(54) COMPOSITIONS AND METHODS FOR IMPROVING THE APPEARANCE OF THE SKIN

(71) Applicant: ISP INVESTMENTS LLC, Wilmington, DE (US)

(72) Inventors: Feng LING, Shanghai (CN); NING Ling, Shanghai (CN); BU Tianyun, Shanghai (CN)

(73) Assignee: ISP INVESTMENTS LLC, Wilmington, DE (US)

(21) Appl. No.: 18/022,244

(22) PCT Filed: Aug. 19, 2021
(86) PCT No.: PCT/IB2021/057666

§ 371 Date: Feb. 20, 2023

(30) Foreign Application Priority Data
Aug. 24, 2020 (CN) 202010866689.1

(51) Int. Cl. A61K 8/65; A61Q 19/02; A61Q 19/08

(52) U.S. Cl. CPC A61K 8/65; A61Q 19/02; A61Q 19/08; A61K 2800/592

(57) ABSTRACT

The present invention relates to compositions and methods for improving the appearance of the skin. Compositions comprises at least water, glycerin, sodium PCA, erythritol, Carrageenan (Chondrus Crispus extract), xanthan Gum; hydrolyzed collagen, hexapeptide-9 and a physiologically acceptable medium. Methods comprise applying the compositions to the skin to improve the appearance of aging skin, increasing skin moisturization, refining skin pores and decreasing aging skin pigmentation defects.

### TECHNICAL FIELD

The invention relates to skin care compositions and methods for improving the appearance of the skin.

### BACKGROUND

Many consumers are concerned with the appearance characteristics of their skin and the signs of skin aging, such as wrinkles and loss of firmness or freckles, age spots or large skin pores.

The skin is a vital organ composed of several layers (dermis, epidermal layers and stratum corneum), which covers the entire surface of the body and provides protective, sensitive, immune, metabolic or thermoregulatory functions. The skin, like the other organs, is subject to aging.

The skin is subjected to a complex physiological process of aging. Intrinsic or chronological aging is the consequence of a genetically programmed senescence and of biochemical alterations due to endogenous factors. In the skin, aging process is characterized by a slow-down in the regeneration of cells and extracellular matrices, a gradually loss of dermal collagen and elastic fibers resulting in dermal and epidermal atrophy, dryness, a reduction in elasticity and firmness of the skin, the appearance of fine lines and wrinkles hyperpigmentation or hypopigmentation blemishes, age spots, etc. Extrinsic aging, on the other hand, is due to environmental attack such as pollution, sun light irradiation (including UV radiation), diseases, lifestyle, etc. This is superimposed on intrinsic aging at zones which are chronically exposed to these attacks; this is then known as photo-aging.

Other cosmetic skin defects than skin aging can occur to healthy individuals, for example: dry skin, skin pigmentation defects, skin tone defects, enlargement and increasing in the number of pores.

Dry skin is a very common skin condition characterized by a lack of the appropriate amount of water in the most superficial layer of the skin. It may occurs depending on hereditary, acquired or environmental factors regardless aging. For example, in a very dry environment water loss by evaporation from the stratum corneum is significant and can exceed the rate of replacement by transcellular diffusion.

Skin pigmentation defects other than aging pigmentation defects may include for example freckles.

Skin tone defects occurring regardless the aging process may include for example dull complexion due to stress, pollution or sleep deprivation or blotchy complexion due to over-skin exfoliation, use of skin irritating products or playing sports.

Enlargement and increasing in the number of pores may be due for example to overproduction of sebum or increased hair follicle volume.

In that respect, an object of the present invention is to provide a composition for application on the skin surface which is capable of providing antiwrinkle, improving skin firmness and elasticity, moisturizing effects, improving skin tone evenness, refining skin pore and lightening skin pigmentation defects.

### SUMMARY

The invention relates to a skin care composition for improving the appearance of the skin comprising a physiologically acceptable medium and an effective amount of an active ingredient consisting of, based on the total weight of the active ingredient:
between 50% to 99.9%, preferably between 70% and 90% of water
between 5% to 20%, preferably between 10% and 15% of glycerin,
between 0.1% to 5.0%, preferably between 1% and 4% of Sodium PCA,
between 0.1% to 3.0%, preferably between 0.5% and 2.0% of Erythritol,
between 0.1% to 2.0%, preferably between 0.3% and 1.0% of Carrageenan (Chondrus Crispus extract),
between 0.1% to 2.0%, preferably between 0.2% and 1.0% of Xanthan Gum,
between 0.1% to 3.0%, preferably between 0.5% and 2.0% of hydrolyzed

Collagen,
between 0.001% to 0.02%, preferably between 0.002% and 0.01% of Hexapeptide-9.

The composition may optionally comprise as preservatives:
between 0.05% to 0.3%, preferably between 0.1% and 0.2% of Benzoic Acid,
between 0.05% to 0.3%, preferably between 0.1% and 0.2% of Sorbic Acid.

The invention also relates to a method for improving the appearance of the skin, wherein the composition comprising the active ingredient is applied on to the skin surface to provide the following cosmetic effects: improving skin signs of aging like skin elasticity, improving skin firmness, reducing wrinkles, lightening age spots, and also refining skin pore, improving skin tone evenness, lightening the skin.

The invention also relates to a method for improving skin moisturization, wherein the composition comprising the active ingredient is applied onto the skin surface during at least 1 hour.

The invention also relates to a cosmetic method for increasing the expression of collagen I, collagen III, collagen IV and collagen XVII in skin cells, wherein the composition comprising the active ingredient is applied onto the skin surface during at least 48 hours.

### BRIEF DESCRIPTION OF THE DRAWINGS

For the purpose of illustrating aspects of the present invention, there are depicted in the drawing's certain embodiments of the invention. However, the invention is not limited to the precise arrangements and instrumentalities of the embodiments depicted in the drawings.

Further embodiments of the present invention can be understood with the appended figures.

FIG. 1 shows the collagen I immunofluorescent strength of ex vivo human skin treated by the composition according to the present invention and a placebo.

FIG. 2 shows the collagen III immunofluorescent strength of ex vivo human skin treated by the composition according to the present invention and a placebo.

FIG. 3 shows the test result of human skin hydration after the treatment with the composition according to the present invention and a placebo.

FIG. 4 shows the test result of human skin hydration after the treatment with the composition according to the present invention and a placebo.

FIG. 5 shows the test result of human skin elasticity after the treatment with the composition according to the present invention and a placebo.

FIG. 6 shows the test result of human skin elasticity after the treatment with the composition according to the present invention and a placebo.

FIG. 7 shows the test result of human skin elasticity after the treatment with the composition according to the present invention and a placebo.

FIG. 8 shows the test result of human skin elasticity after the treatment with the composition according to the present invention and a placebo.

FIG. 9 shows the test result of human skin firmness after the treatment with the composition according to the present invention and a placebo.

FIG. 10 shows the test result of human skin crow's feet after the treatment with the composition according to the present invention and a placebo.

FIG. 11 shows the test result of human skin pore number after the treatment with the composition according to the present invention and a placebo.

FIG. 12 shows the test result of human skin pore area after the treatment with the composition according to the present invention and a placebo.

FIG. 13 shows the test result of human skin redness after the treatment with the composition according to the present invention and a placebo.

FIG. 14 shows the test result of human skin age spot after the treatment with the composition according to the present invention and a placebo.

FIG. 15 shows the test result of human skin tone after the treatment with the composition according to the present invention and a placebo.

### DETAILED DESCRIPTION

The present disclosure provides a new skin care composition for improving the appearance of the skin. It was surprisingly found that the composition has favorable effects with regards to improving skin elasticity, improving skin firmness, reducing wrinkles, refining skin pore, improving skin tone evenness and lightening age spots.

### Active Ingredient

The active ingredient of the composition comprises (a) a synthetic hexapeptide of sequence (Gly-Pro-Gln)₂-NH2 which was disclosed in the patent documents U.S. Pat. No. 7,211,269 and WO2003007905. This peptide was designed for increasing cutaneous cell adhesion and has effects on reducing wrinkles and repairing skin. This peptide has the INCI name Hexapeptide-9 Hexapeptide-9 is sold by Ashland under the commercial name Collaxyl™.

The active ingredient of the composition comprises also (b) the following mixture of compounds: water, glycerin, sodium PCA (Sodium Pyrrolidonecarboxylate), erythritol, carrageenan (Chondrus crispus extract) and xanthan Gum.

This mixture was described to hydrate skin effectively.

The active ingredient of the composition comprises finally (c) hydrolyzed collagen. These collagen oligopeptides were associated with increase in collagen I and collagen III and to boost hyaluronic acid expression in human skin.

As an example of hydrolyzed collagen, it can be cited low molecular weight collagen oligopeptides of marine origin and more particularly, Marine Hydrolyzed Collagen LMW™ sold by Ashland under the INCI name: Water (and) Hydrolyzed Collagen.

In one aspect, the present disclosure provides a skin care composition comprising a physiologically acceptable medium and an effective amount of an active ingredient consisting of the following ingredients with concentration expressed in weight percent with regards to the total weight of the active ingredient:
between 50% to 99.9%, preferably between 70% and 90% of water
between 5% to 20%, preferably between 10% and 15% of glycerin,
between 0.1% to 5.0%, preferably between 1% and 4% of Sodium PCA,
between 0.1% to 3.0%, preferably between 0.5% and 2.0% of Erythritol,
between 0.1% to 2.0%, preferably between 0.3% and 1.0% of Carrageenan (Chondrus Crispus extract),
between 0.1% to 2.0%, preferably between 0.2% and 1.0% of Xanthan Gum,
between 0.1% to 3.0%, preferably between 0.5% and 2.0% of Hydrolyzed Collagen,
between 0.001% to 0.02%, preferably between 0.002% and 0.01% of Hexapeptide-9.
optionally between 0.05% to 0.3%, preferably between 0.1% and 0.2% of Benzoic Acid,
optionally between 0.05% to 0.3%, preferably between 0.1% and 0.2% of Sorbic Acid.

In on a embodiment, the hydrolyzed Collagen is a low molecular weight hydrolyzed collagen of marine origin.

In another embodiment, the composition comprises an active ingredient consisting of the compounds as listed above:
81.696% of water,
12% of Glycerin;
2.7% of Sodium PCA;
0.9% of Erythritol;
0.6% of Carrageenan (Chondrus Crispus extract);
0.3% of Xanthan Gum;
0.18% of Benzoic Acid;
0.12% of Sorbic Acid;
1.5% of Hydrolyzed Collagen;
0.004% of Hexapeptide-9.

The procedure to prepare the active ingredient comprises the following steps: add water in a clean vessel, heat to 80-85 degree C. and keep 30 minutes, then cool down to room temperature. Begin mixing, in the vortex slowly sprinkle Xanthan Gum, Carrageenan, mix until fully dissolved. Add in glycerin, sodium PCA, Erythritol, Benzoic Acid, Sorbic Acid, Hexapeptide-9 and Hydrolyzed marine Collagen, stir well until uniform after each addition.

One advantage of the present active ingredient is to provide a ready-to-formulate complex composition. The active ingredient is water soluble, easy to formulate into water based formulations (facial mask, serum, clear lotion, etc.) and emulsion type of formulations.

### Definitions

Whenever a term is identified by reference to a range, the range will be understood to explicitly disclose every element thereof. As a non-limiting example, a range of 1-10% will be understood to include 1%, 2%, 3%, 4%, 5%, 6%, 7%, 8%, 9%, and 10%, and all values between 1 and 10%.

Where two or more substituents are referred to as being “selected from” a group of enumerated alternatives, it is meant that each substituent can be any element of that group, independent of the identity of the other substituents.

As used herein, “% refers to % by weight, that is the weight percent of a component in relation to the total weight of the composition (i.e., including any carriers, vehicles, solvents, fillers, or other components added before application to the skin) unless otherwise provided.

All terms used herein are intended to have their ordinary meaning unless otherwise provided. For the purposes of describing and claiming the present invention, the following terms are defined:

The compositions described and used in the present disclosure can “comprise”, “consist essentially of”, or “consist of”, the essential components as well as optional ingredients described herein. As used herein, “consisting essentially of” means that the composition or component may include additional ingredients, but only if the additional ingredients do not materially alter the basic and novel characteristics of the claimed compositions or methods.

“Apply” or “Application” as used in reference to a skin care composition means to apply or spread the compositions of the present invention onto a human skin surface such as the epidermis.

“Physiologically acceptable medium” as used herein means, in the skin care compositions of the disclosure, a medium suitable for use in contact with human skin tissue without toxicity, incompatibility, instability, allergic response and the like, capable of being applied to the skin, integuments or lips of the face or the body of mammals or human beings.

“Improving the appearance of aging skin” as used herein means, means effecting a visually and/or tactilely perceptible positive change, or benefit, in skin appearance and/or feel.

“improving skin signs of aging” as used herein, means preventing or delaying the appearance of aging skin. Benefits that may be provided include, but are not limited to, one or more of the following: Reducing the appearance of wrinkles and coarse deep lines, fine lines, crevices, preventing loss of skin elasticity and firmness, improving the appearance of age spots or any other pigmentation defects related to skin aging, etc. The signs of aging also include any degradation of the skin following exposure to UV rays, such as the premature appearance of fine lines around the eyes and the mouth, and expression lines on the face and thinning of the lips. What is also meant is any internal modification of the skin which does not manifest itself systematically in the form of a modified external aspect, for example the thickening of the vessel walls, the modification of the shape of the fibroblasts, the slowing of collagen synthesis and a disorganization of collagen fibrils, an accumulation of abnormal and amorphous material containing elastin (solar elastosis).

“skin pore refining” or “refine skin pore” as used herein means decreasing the size of skin pore, visible pore number and pore area in treated zone.

“improving skin tone evenness” as used herein means any modification leading to the improvement of the uniformity of skin coloration, decreasing skin redness. “Improving skin tone evenness”, also includes the improvement of dull and blotchy complexion.

“skin lightening” means decreasing the intensity in color of hyperpigmented skin areas, such as freckles, age spots, solar lentigines or any other skin pigmentation defect.

“Active ingredient” as used herein means the following mixture of components: water, glycerin, sodium PCA (Sodium Pyrrolidonecarboxylate), erythritol, carrageenan (Chondrus crispus extract, xanthan gum, hydrolyzed collagen, Hexapeptide-9 and optionally benzoic acid and sorbic acid. It may be equally named Natriance™ Ageless or Natriance ageless.

“additional active agent” as used herein means any cosmetic agent having cosmetic effects similar and/or complementary to the active ingredient disclosed herein.

“Effective amount” as used herein means an amount of the composition sufficient to significantly induce a positive appearance and/or feel benefit, but low enough to avoid serious side effects (i.e., to provide a reasonable benefit to risk ratio, within the scope of sound judgment of the skilled artisan).

“Preservative” as used herein means a substance used to prevent microbial growth or undesirable chemical changes and suitable for cosmetic compositions, for example benzoic acid and sorbic acid.

“Skin moisturizing” as used herein, means the water content of the upper layer of the skin.

As used herein, the term “elasticity and firmness” means the ability of skin to resist and recover from external physical stress such as gravity.

As used herein, the term “skin erythema” means skin redness, normally caused by skin sensitivity, occurring in healthy individuals and not linked to pathological disorders.

It is obvious that the invention concerns mammals in general, and more specifically human beings.

### Skin Care Composition

In one embodiment the composition comprises the active ingredient Natriance ageless at a concentration ranging from 1 to 10% (w/w) in a physiologically acceptable medium.

In another embodiment the composition comprises the active ingredient Natriance ageless at a concentration ranging from 1 to 5% (w/w) in a physiologically acceptable medium.

The compositions usable in accordance with this disclosure are suitable for topical application and the formulation of the compositions will be adapted by a person skilled in the art.

Topical application as used herein has the meaning of applying or spreading the skin care composition on the skin surface or on a mucosa.

The skin care compositions could be in particular in the form of an aqueous, hydro-alcoholic or oily solution, an oil-in-water emulsion, or water-in-oil emulsion. They could also be presented in the form of suspensions or even powders, suitable for application to the skin, mucosae, lips, and/or hair.

These compositions may be fluid to a greater or lesser extent and may also have the appearance of a cream, a lotion, a milk, a serum, a pomade, a gel, a paste or a foam. They may also be presented in solid form, as a stick, or may be applied to the skin in aerosol form.

Examples of a physiologically acceptable medium commonly used in the envisaged field of application include the additives necessary for the formulation, such as solvents, thickeners, diluents, antioxidants, colorants, sun filters, self-tanning agents, pigments, fillers, preservatives, perfumes, odor absorbers, essential oils, vitamins, essential fatty acids, surfactants, filmogenic polymers, etc.

In any case, a person skilled in the art will ensure that these additives as well as their proportions are selected to as not to compromise the advantageous properties sought of the composition according to the invention. These additives may correspond, for example, to 0.01 to 20% of the total weight of the composition. When the composition according to the invention is an emulsion, the oil phase may represent from 5 to 80% by weight, and preferably from 5 to 50% by weight in relation to the total weight of the composition. The emulsifiers and co-emulsifiers used in the composition are selected from those used conventionally in the field in question. For example, they can be used in a proportion ranging from 0.3 to 30% by weight in relation to the total weight of the composition.

In accordance with a further advantageous embodiment of the disclosure, the skin care composition can be encapsulated or included in a cosmetic vector, such as liposomes or any other nanocapsule or microcapsule used in the field of cosmetics or adsorbed on powdered organic polymers or mineral supports, such as talcs and bentonites.

The skin care compositions may further comprise, at least one other active agent having cosmetic effects similar and/or complementary to those of the invention. As used herein this active ingredient is defined as an “additional active agent”.

For example, the additional active ingredient(s) may be selected from: anti-ageing, firming, brightening, hydrating, draining, microcirculation-promoting, exfoliating, desquamating, extracellular matrix-stimulating, energy metabolism-activating, antibacterial, anti-fungal, soothing, anti-radical, anti-UV, anti-acne, anti-inflammatory substances, substances inducing a sensation of heat or a sensation of freshness, and slimming substances.

Such additional active agent can be selected from the groups comprising:
vitamin A and in particular retinoic acid, retinol, retinol propionate, retinol palmitate;
vitamin B3 and more particularly niacinamide, tocopherol nicotinate;
vitamin B5, vitamin B6, vitamin B12, panthenol;
vitamin C, in particular ascorbic acid, ascorbyl glucoside, ascorbyl tetrapalmitate, magnesium and sodium ascorbyl phosphate;
vitamins E, F, H, K, PP, coenzyme Q10;
inhibitors of metalloproteinase, or an activator of TIMPs;
DHEA, its precursors and derivatives;
amino acids such as arginine, ornithine, hydroxyproline, hydroxyproline dipalmitate, palmitoylglycine, hydroxylysine, methionine and derivatives thereof, N-acyl amino acid compounds;
natural or synthetic peptides, including the di-, tri-, tetra-, penta- and hexapeptides and their lipophilic and isomeric derivatives and their derivatives complexed with other species, such as a metal ion (for example copper, zinc, manganese, magnesium, and others). Examples include the peptides known commercially by the name MATRIXYL®, ARGIRELINE®, CHRONOGEN™, LAMINIXYL IS™, PEPTIDE Q10™, PEPTIDE VINCI 01™ (patent FR2837098, ASHLAND®), PEPTIDE VINCI 02™ (patent FR2841781, ASHLAND®), ATPeptide™ (patent FR2846883, ASHLAND®) or also the synthetic peptide of sequence Arg-Gly-Ser-NH2, sold under the name ATPeptide™ by ASHLAND®;
the extract of Artemia salina, sold under the name GP4G™ (FR2817748) ASHLAND®;
plant peptide extracts, such as extracts of flax (Lipigenine™, patent FR2956818, ASHLAND®), extracts of soybean, spelt, vine, rape, flax, rice, corn, pea;
yeast extracts, for example Dynagen™, (patent FR2951946, ASHLAND®) or Actopontine™ (patent FR2944526, ASHLAND®);
dehydroacetic acid (DHA);
phytosterols of synthetic or natural origin;
salicylic acid and derivatives thereof, alpha- and beta-hydroxy acids, silanols;
amino sugars, glucosamine, D-glucosamine, N-acetyl glucosamine, N-acetyl-D-glucosamine, mannosamine, N-acetyl mannosamine, galactosamine, N-acetyl galactosamine;
extracts of polyphenols, isoflavones, flavonoids, such as extracts of grape, extracts of pine, extracts of olive;
lipids, such as the ceramides or phospholipids, oils of animal origin, such as squalene or squalane; plant oils, such as sweet almond oil, coconut oil, castor oil, jojoba, oil, olive oil, rape oil, peanut oil, sunflower oil, wheat germ oil, maize germ oil, soybean oil, cotton oil, alfalfa oil, poppy oil, pumpkin oil, evening primrose oil, millet oil, barley oil, rye oil, safflower oil, passiflora oil, hazelnut oil, palm oil, apricot kernel oil, avocado oil, calendula oil; ethoxylated plant oils, shea butter;
all UV screens and sun filters;
cyclic AMP and derivatives thereof, substances that are activators of the adenylate cyclase enzyme and substances that are inhibitors of the phosphodiesterase enzyme, the extract of Centella asiatica, asiaticoside and asiatic acid, methylxanthines, theine, caffeine and derivatives thereof, theophylline, theobromine, forskolin, esculin and esculosid, ACE inhibitors, the peptide Val-Trp, inhibitors of neuropeptide Y, enkephalin, extract of Ginkgo biloba, extract of dioscorea, rutin, extract of yerba mate, extract of guarana, oligosaccharides, polysaccharides, carnitine, extract of ivy, extract of fucus, hydrolysed extract of Prunella vulgaris, hydrolysed extract of Celosia cristata, extract of Anogeissus leiocarpus, extract of leaves of Manihot utilissima, palmitoyl carnitine, carnosine, taurine, extract of elder, extracts of seaweed, such as the extract of Palmaria Palmata.

### Cosmetic Method for Improving the Appearance of the Skin

In accordance to the present disclosure, it was unexpectedly found that the composition described herein improves several aspects of the skin selected from: increases skin elasticity, increases skin firmness, refines skin pore, reduces skin wrinkles, improves skin tone evenness and lightens skin and age spots.

In one aspect the disclosure relates to a cosmetic method for improving the appearance of the skin, wherein the composition described above is applied on the skin surface and wherein the effects on the skin include the following skin signs of aging: improving skin elasticity, improving skin firmness, reducing wrinkles, lightening age spots.

In this aspect, advantageously the composition comprises the active ingredient Natriance ageless at a concentration ranging from 1 to 5% (w/w) in a physiologically acceptable medium and is applied during at least 28 days.

In this aspect, advantageously the composition comprises the active ingredient Natriance ageless at a concentration of 5% (w/w) in a physiologically acceptable medium and is applied during at least 28 days.

In another embodiment the disclosure relates to a cosmetic method for improving the appearance of the skin, said method comprising applying to the skin surface the composition, described above wherein the effects on the skin are selected among: refining skin pore, improving skin tone evenness and lightening the skin.

In this aspect, advantageously the composition comprises the active ingredient Natriance ageless at a concentration ranging from 1 to 5% (w/w) in a physiologically acceptable medium and is applied during at least 28 days.

In this aspect, advantageously the composition comprises the active ingredient Natriance ageless at a concentration of 5% (w/w) in a physiologically acceptable medium and is applied during at least 28 days.

In another aspect the disclosure relates to a cosmetic method for improving skin moisturization comprising applying to the skin surface the composition described above.

In this aspect, advantageously the composition comprises the active ingredient Natriance ageless at a concentration ranging from 1 to 5% (w/w) in a physiologically acceptable medium and is applied from 1 to 5 hours.

In this aspect, advantageously the composition comprises the active ingredient Natriance ageless at a concentration of 5% (w/w).

In another aspect the disclosure relates to a cosmetic method for increasing the expression of Collagen I, collagen III, Collagen IV and collagen XVII comprising applying the composition described above twice a day for 48 hours.

In this aspect advantageously the composition comprises the active ingredient Natriance ageless at a concentration ranging from 1 to 5% (w/w) in a physiologically acceptable medium.

### Example 1: Preparation of the Active Ingredient Natriance Ageless

Add water in a clean vessel, heat to 80-85 degree C. and keep 30 minutes, then cool down to room temperature. Begin mixing, in the vortex slowly sprinkle Xanthan Gum, Carrageenan, mix until fully dissolved. Add in glycerin, sodium PCA, Erythritol, Benzoic Acid, Sorbic Acid, Hexapeptide-9 and Hydrolyzed Collagen, stir well until uniform after each addition.

Listing and concentration of each compound used for preparing the active ingredient Natriance Ageless are displayed in the below Table 1

TABLE 1

| | content |
|---|---|
| INCI name | %(w/w) |
| Aqua | 81.696 |
| Glycerin | 12 |
| Sodium PCA (Sodium | 2.7 |
| Pyrrolidonecarboxylate) | |
| Erythritol | 0.9 |
| Carrageenan (Chondrus | 0.6 |
| Crispus extract) | |
| Xanthan Gum | 0.3 |
| Benzoic Acid | 0.18 |
| Sorbic Acid | 0.12 |
| Hydrolyzed Collagen | 1.5 |
| Hexapeptide-9 | 0.004 |

Typical properties of the obtained active ingredient Natriance Ageless: Appearance: Slightly haze viscous liquid
pH value: 4.0-5.0 (as is)
Viscosity: 3,000-20,000 cps (Brookfield RVDV-II+, 05 #@10 rpm)

### Example 2: Immunostaining of Collagen I and III in Ex Vivo Skin

Protocol:

Normal human skin biopsies of 6 mm of diameter were maintained ex vivo in a specific culture medium (DMEM at 1 g/L, HAMF12, fetal calf serum and antibiotics). Biopsies were treated with the active ingredient of example 1 diluted in polybutylene succinate (PBS) at concentrations of 1%, 3% and 5% twice a day for 48 hours. The diluted active ingredient was applied onto the surface of the skin biopsie. The placebo comparison is performed by applying PBS 1× (pure polybutylene succinate) in place of the diluted active ingredient.

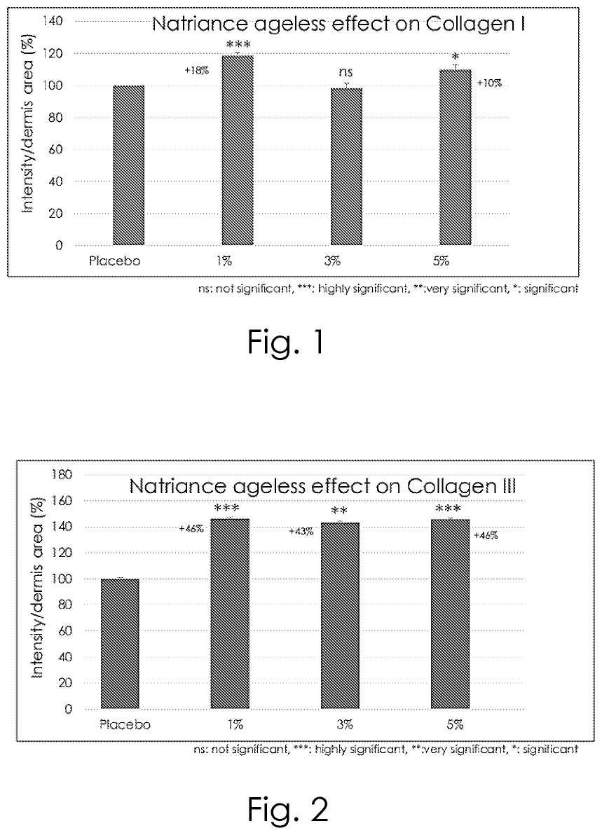

For immunolabelling by collagen land collagen III antibodies, tissues were fixed and embedded in paraffin. Embedded skin biopsies were then cut and sections were deparaffinized and rehydrated. Then, an unmasking protocol was performed before applying a specific anti-collagen I antibody (abcam, ab34710, rabbit polyclonal) or a specific anti-collagen III antibody (Tebu-Rockland, 600-401-105, rabbit polyclonal), and then a secondary suitable antibody, coupled with a fluorescent dye. After mounting in a particular medium, the slides were observed by epifluorescence microscope (Zeiss Axiovert 200M microscope), and statistical analysis was conducted by student's T test. The test results are shown in FIG. 1 and FIG. 2.

Results:

FIG. 1 shows the collagen I immunofluorescent strength of ex vivo human skin treated by the composition according to the present invention and a placebo. As shown in FIG. 1, when compared with the treatment with the placebo for 48 hours, the treatments with the active ingredient of example 1 diluted at concentrations of 1 and 5% for 48 hours showed respectively a highly significant (+18%) and significant increase (+10%) in Collagen I expression in ex vivo skin biopsies. The treatment with the active ingredient of example 1 diluted at concentrations of 3% shows no significant increase when compared with the treatment with the placebo, which may be due to operation error.

FIG. 2 shows the collagen III immunofluorescent strength of ex vivo human skin treated by the composition according to the present invention and a placebo. the treatments with the active ingredient of example 1 for 48 hours showed also a highly significant increase at concentrations of 1% and 5% and a very significant increase at 3% in collagen III expression in ex vivo skin biopsies.

Conclusion:

The active ingredient of example 1, through the stimulation of Collagen I and collagen III, improved the extracellular matrix structure important to fight aging.

### Example 3: Clinical Study on Skin Moisturization

Protocol:

24 Asian volunteers, both male and female, double-blind test, dry skin, the hydration value on the volar side of forearm is below 40. Choose 2 zones of 3 cm*5 cm on the volar forearms, test zones are treated with test products as shown in Table 2 (see formulation, 5% Natriance Ageless, as disclosed in example 1, and placebo) as per randomization and at a dose of 2 mg/cm². Test skin hydration (Corneometer CM825) and skin trans epidermal water loss (TEWL, Tewameter TM300) at 0 hour, 1 hour, 3 hours and 5 hours (T0, T1, T3, T5). Use Student's Test (one-tailed) for statistical analysis.

TABLE 2
Test formulation

| | | Active | |
|---|---|---|---|
| | | ingredient | Placebo |
| Ingredients (Trade Name \| INCI) | | % w/w | |
| Phase A | | | |
| Purified Water | WATER | To 100 | To 100 |
| EDTA-2Na | DISODIUM EDTA | 0.05 | 0.05 |
| Ashland 980 Polymer | CARBOMER | 0.40 | 0.40 |
| Phase B | | | |
| NaOH (10% water | SODIUM HYDROXIDE | 0.71 | 0.71 |
| solution) | | | |
| Phase C | | | |
| Methylparaben | METHYLPARABEN | 0.20 | 0.20 |
| Optiphen PO | PHENOXYETHANOL | 0.30 | 0.30 |
| Ethylparaben | ETHYLPARABEN | 0.10 | 0.10 |
| Phase D | | | |
| Natriance ™Ageless | WATER (AND) GLYCERIN (AND) SODIUM | 5.00 | 0.00 |
| (Active ingredient of | PCA(AND) ERYTHRITOL(AND) HYDROLYZED | | |
| example 1) | COLLAGEN(AND) CHONDRUS CRISPUS | | |
| | (CARRAGEENAN) (AND) XANTHAN GUM(AND) | | |
| | HEXAPEPTIDE-9 | | |
| Total | | 100 | 100 |

Preparing procedure of the tested product:
1. Add water in a beaker and heat to 65° C., add Phase C, and mix until uniform.
2. Cool the batch to room temperature, spread CARBOMER and DISODIUM EDTA in the vortex, and mix until uniform.
3. Add 10% NaOH water solution with stirring, mix until uniform.
4. Add Phase D ingredient and mix until uniform.

Results:

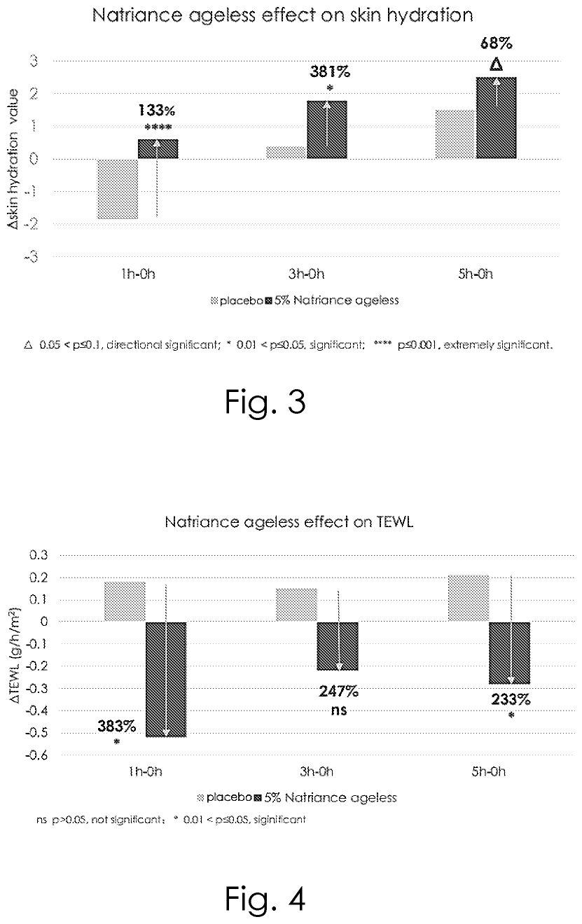

FIG. 3 shows the test result of human skin hydration after the treatment with the composition according to the present invention and a placebo. As shown in FIG. 3, when compared with the treatment with the placebo, 5% Natriance Ageless (Active ingredient of example 1) increases skin hydration at 1 h, 3 h and 5 h statistically significant. ΔSkin hydration value is obtained by substracting the hydration value at 0 h from the hydration value at 1 h, 3 h and 5 h. The percentage in FIG. 3 is obtained by the difference value between the ΔSkin hydration value of the 5% Natriance Ageless and the ΔSkin hydration value of the placebo, divided by the ΔSkin hydration value of the placebo. “p” is obtained by dividing the standard deviation by the average value.

FIG. 4 shows the test result of human skin hydration after the treatment with the composition according to the present invention and a placebo. As shown in FIG. 4, when compared with the treatment with the placebo, 5% Natriance Ageless reduces TEWL at 1 h and 5 h statistically significant. ΔTEWL is obtained by substracting the ΔTEWL at 0 h from the ΔTEWL at 1 h, 3 h and 5 h. The percentage in FIG. 4 is obtained by the difference value between the ΔTEWL of the 5% Natriance Ageless and the ΔTEWL of the placebo, divided by the ΔSkin hydration value of the placebo. “p” is obtained by dividing the standard deviation by the average value.

Conclusion:

Natriance Ageless (Active ingredient of example 1) can improve instant skin hydration and through strenthening skin barrier function can effectively reduce transepidermal water loss.

### Example 4: Clinical Study to Determine Skin Benefits of Natriance Ageless

Protocol:

30 Asian candidates (with wrinkles and slack skin, aging 47.97±5.71), double-blind test on face. Half face are treated with products as shown in Table 3 (see formulation, 5% Natriance Ageless, the Active ingredient of example 1 and placebo) as per randomization at a dose of 2 mg/cm². Take full face imaging with Visia CR, take skin crow's feet wrinkles photos with Primos, test skin elasticity and firmness with Cutometer at visits of Day 0, Day 7, Day 14 and Day 28. Use Student's Test (one-tailed) for statistical analysis.

TABLE 3
Test formulation:

| | | #20039A | |
|---|---|---|---|
| | | Active | #20039P |
| | | ingredient | Placebo |
| Ingredients (Trade Name \| INCI) | | % w/w | |
| Phase A | | | |
| Purified Water | WATER | To 100 | To 100 |
| EDTA-2Na | DISODIUM EDTA | 0.05 | 0.05 |
| Natrosol ™ Plus 330 cs | CETYL HYDROXYETHYLCELLULOSE | 0.20 | 0.20 |
| Butylene Glycol | BUTYLENE GLYCOL | 3.00 | 3.00 |
| Phase B | | | |
| Prolipid ™ 141 | GLYCERYL STEARATE (AND) BEHENYL | 1.00 | 1.00 |
| | ALCOHOL (AND) PALMITIC ACID (AND) | | |
| | STEARIC ACID (AND) LECITHIN (AND) LAURYL | | |
| | ALCOHOL (AND) MYRISTYL ALCOHOL (AND) | | |
| | CETYL ALCOHOL | | |
| Refined Shea Butter | BUTYROSPERMUM PARKII (SHEA BUTTER) | 1.10 | 1.10 |
| Ceraphyl ™ ODS | OCTYLDODECYL STEARATE | 3.50 | 3.50 |
| Phase C | | | |
| Rapithix ™ A-100 | SODIUM POLYACRYLATE | 1.00 | 1.00 |
| Phase D | | | |
| Lubrajel ™ CG | GLYCERIN (AND) GLYCERYL | 5.00 | 5.00 |
| | ACRYLATE/ACRYLIC ACID COPOLYMER (AND) | | |
| | PROPYLENE GLYCOL | | |
| Natriance ™Ageless | WATER (AND) GLYCERIN (AND) SODIUM | 5.00 | 0.00 |
| (Active ingredient of | PCA(AND) ERYTHRITOL(AND) HYDROLYZED | | |
| example 1) | COLLAGEN(AND) CHONDRUS CRISPUS | | |
| | (CARRAGEENAN) (AND) XANTHAN GUM(AND) | | |
| | HEXAPEPTIDE-9 | | |
| KF-96A-6T | DIMETHICONE | 1.00 | 1.00 |
| Optiphen ™ GP | PHENOXYETHANOL(AND)PROPANEDIOL(AND)PROPYLENE | 0.80 | 0.80 |
| | CARBONATE (AND) | | |
| | CAPRYLHYDROXAMIC ACID(AND)o-CYMEN-5-OL | | |
| Total | | | |

Procedure:
1. Add Phase A ingredients in beaker and mix with heating to 70° C. until uniform.
2. Add Phase B ingredients in another, mix with heating to 70° C. until uniform.
3. Add Phase C ingredient in Phase B with homogenizing, for 10 seconds.
4. Add Phase B+C ingredient in Phase A with homogenizing, for 3 mins. Begin to cool the batch.
5. When the batch is cooled to 35-40° C., add Phase D ingredients one by one and mix until uniform,

Results:

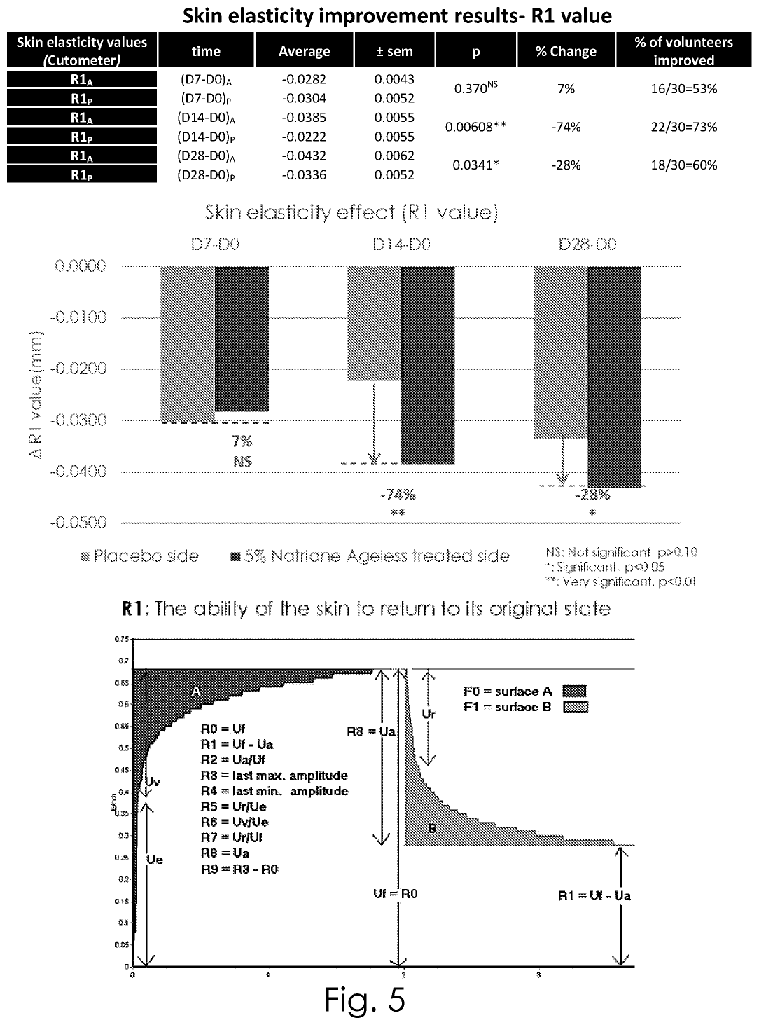

FIG. 5 shows the test result of human skin elasticity after the treatment with the composition according to the present invention and a placebo. Cutometer data showed that 5% Natriance Ageless (Active ingredient of example 1) can improve the ability of skin to return to its original state (R1) at day 14 and day 28 statistically significant.

In FIG. 5 to FIG. 10, “p” is obtained by dividing the standard deviation by the average value. When illustrating the skin elasticity value and time, the suffix “P” indicates the placebo, and “A” indicates 5% Natriance Ageless.

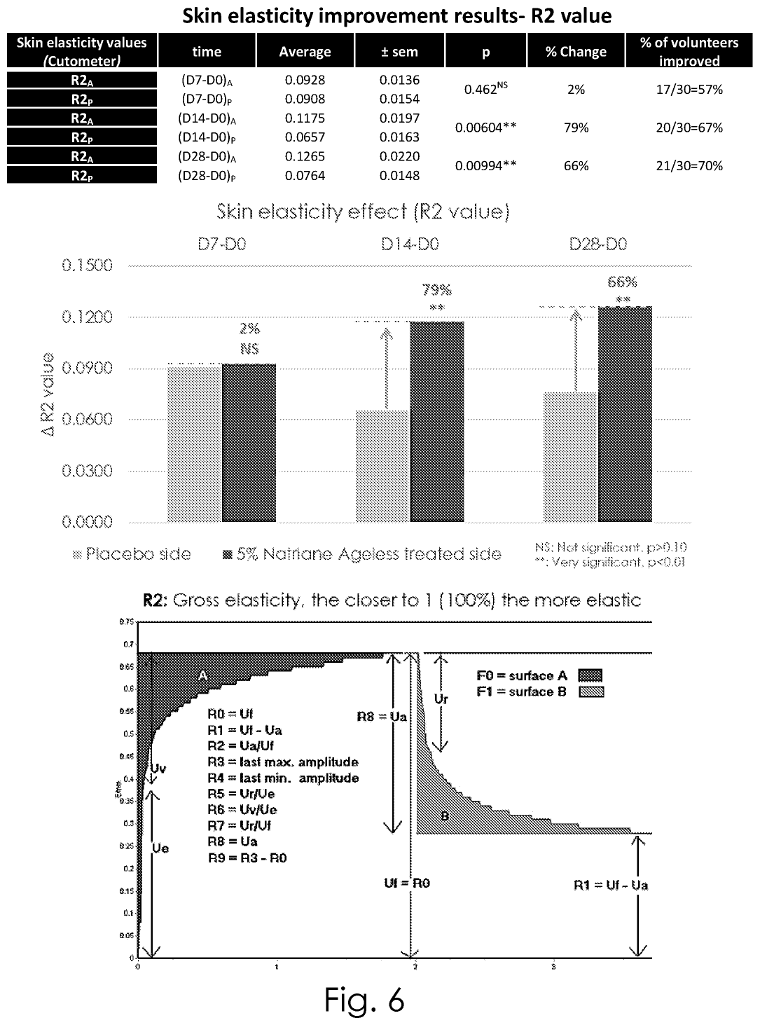

FIG. 6 shows the test result of human skin elasticity after the treatment with the composition according to the present invention and a placebo. Cutometer data showed that 5% Natriance Ageless (Active ingredient of example 1) can improve the gross elasticity (R2) at day 14 and day 28 statistically significant.

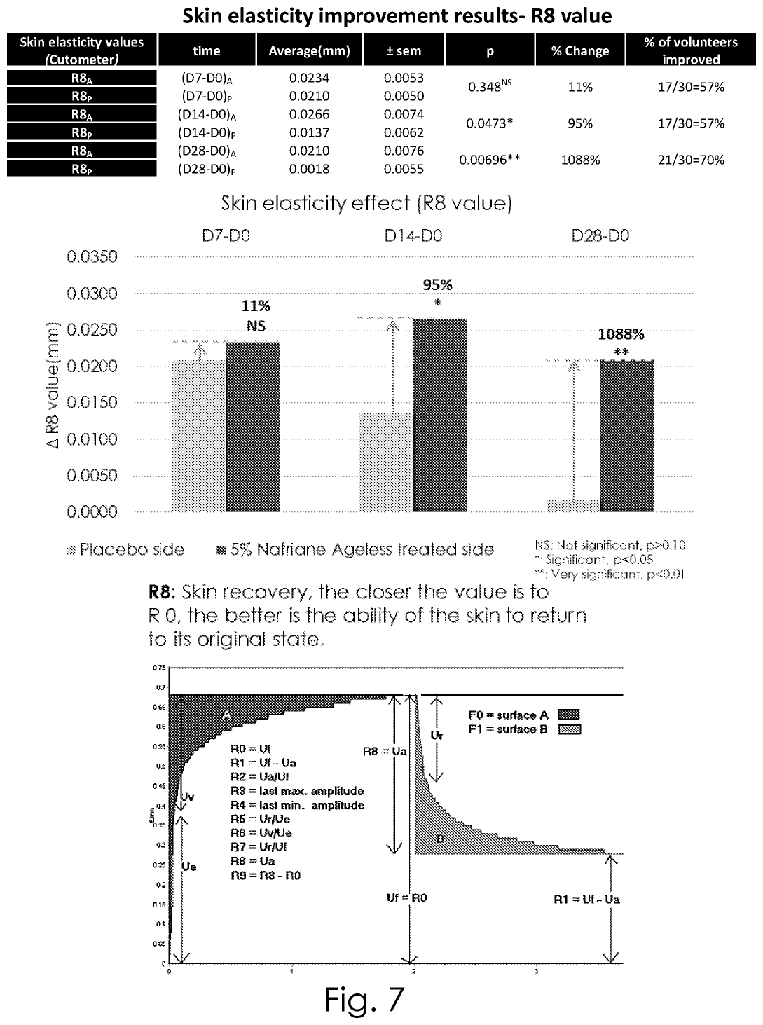

FIG. 7 shows the test result of human skin elasticity after the treatment with the composition according to the present invention and a placebo. Cutometer data showed that 5% Natriance Ageless (Active ingredient of example 1) can improve skin recovery ability (R8, as shown in FIG. 7) at day 14 and day 28 significant.

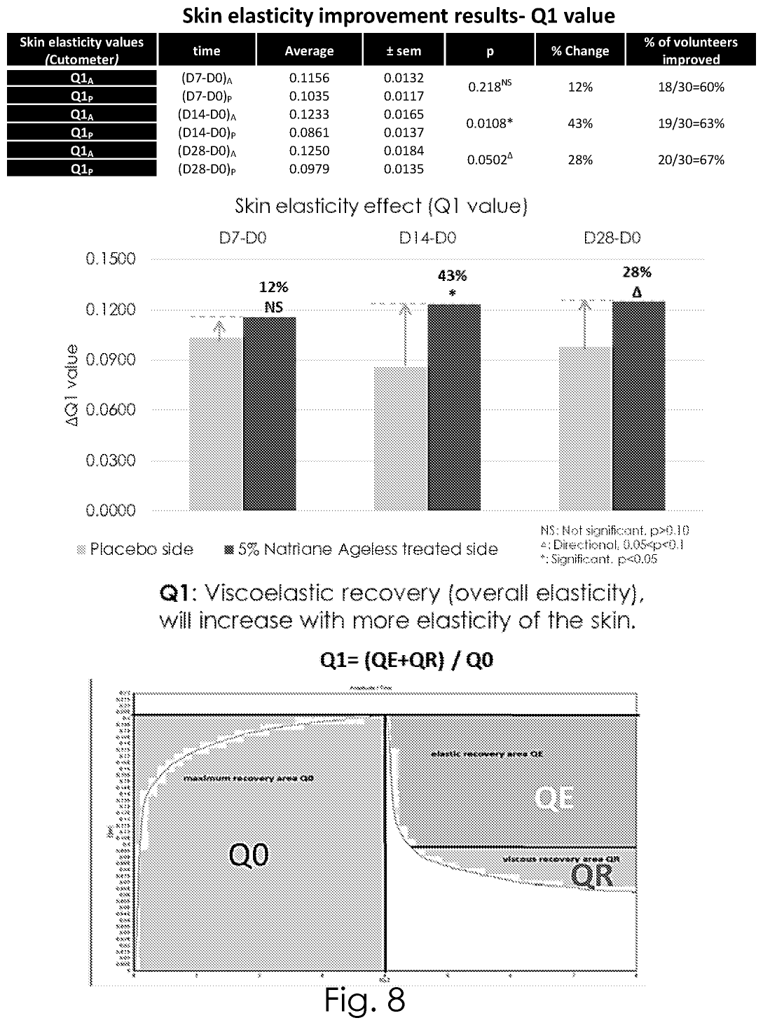

FIG. 8 shows the test result of human skin elasticity after the treatment with the composition according to the present invention and a placebo. Cutometer data showed that 5% Natriance Ageless (Active ingredient of example 1) can improve overall elasticity (viscoelastic recovery ability, Q1) at day 14 and day 28 statistically significant.

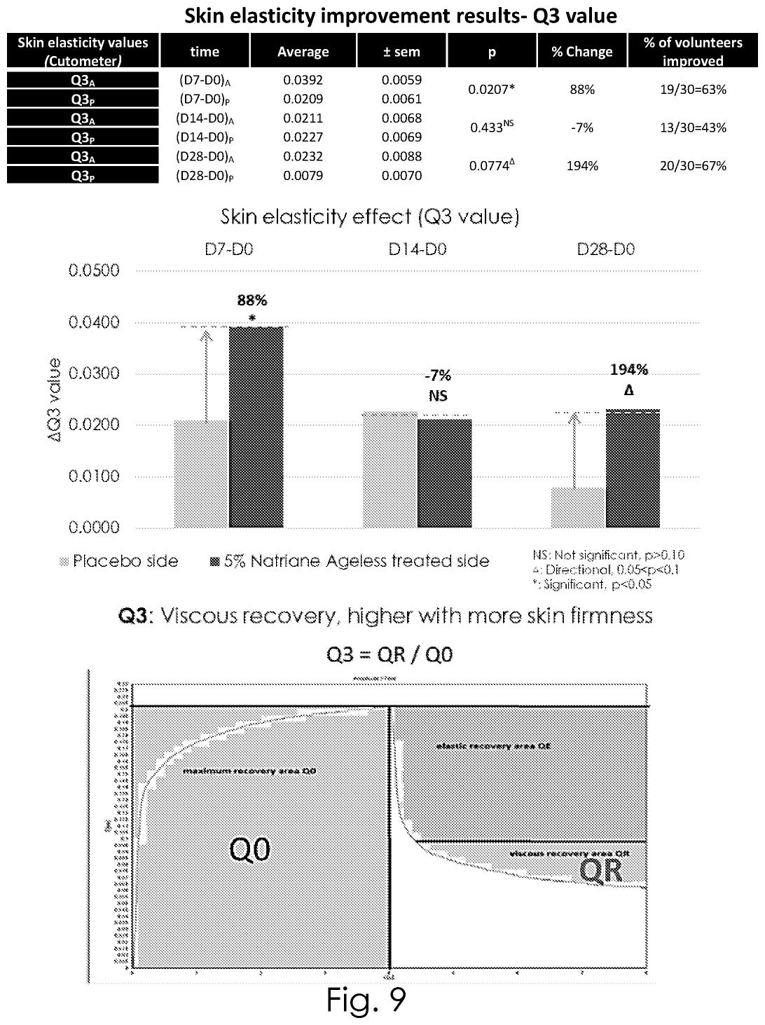

FIG. 9 shows the test result of human skin firmness after the treatment with the composition according to the present invention and a placebo. Cutometer data showed that 5% Natriance Ageless (Active ingredient of example 1) can improve skin firmness (viscous recovery ability, Q3) at day 7 and day 28 statistically significant.

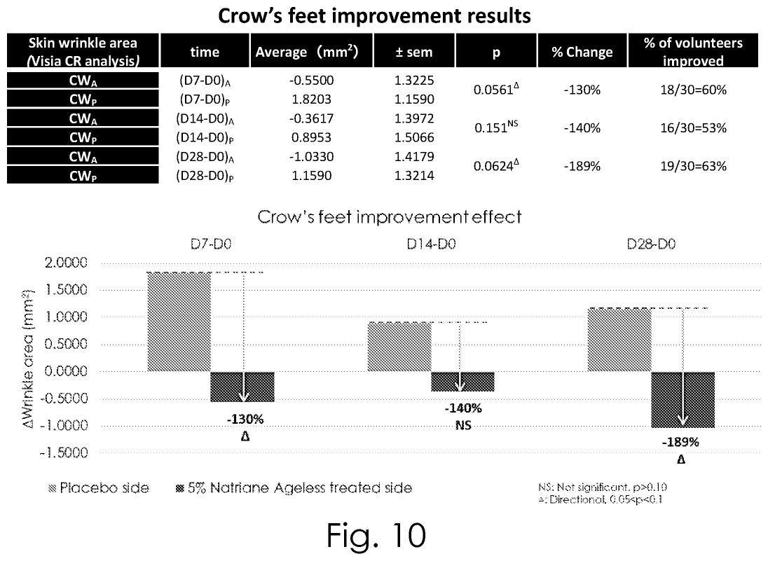

FIG. 10 shows the test result of human skin crow's feet after the treatment with the composition according to the present invention and a placebo. The crow's feet photo taken by Primos showed that 5% Natiance Ageless (Active ingredient of example 1) can reduce crow's feet CW at day 7 and day 28 statistically significant.

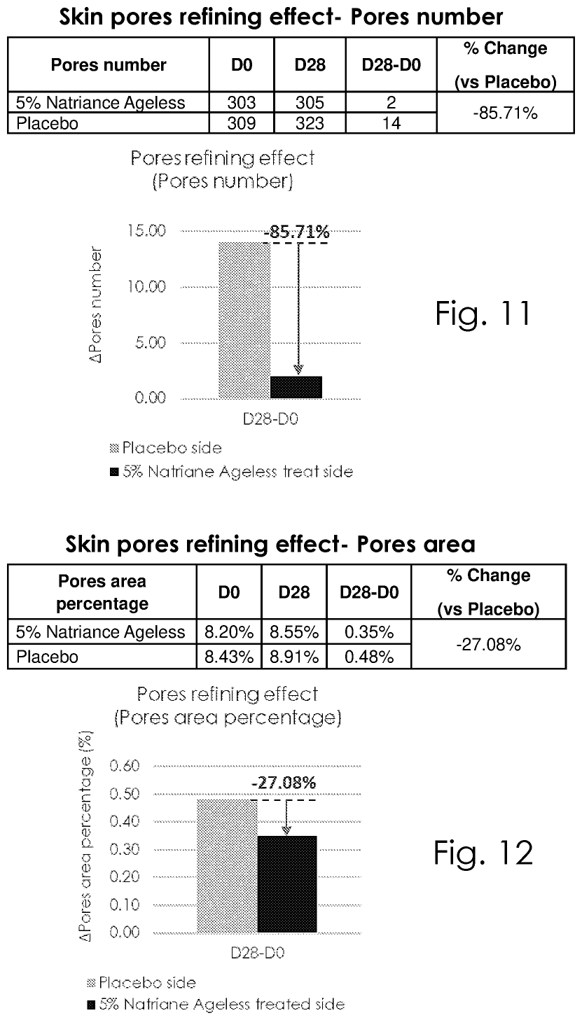

FIG. 11 shows the test result of human skin pore number after the treatment with the composition according to the present invention and a placebo. Visia CR imaging of full face through image analysis showed that 5% Natiance Ageless (Active ingredient of example 1) can provide skin pore refining effect (reduce pore number) after 28 days treatment.

FIG. 12 shows the test result of human skin pore area after the treatment with the composition according to the present invention and a placebo. Visia CR imaging of full face through image analysis showed that 5% Natiance Ageless (Active ingredient of example 1) can provide skin pore refining effect (reduce pore area percentage) after 28 days treatment.

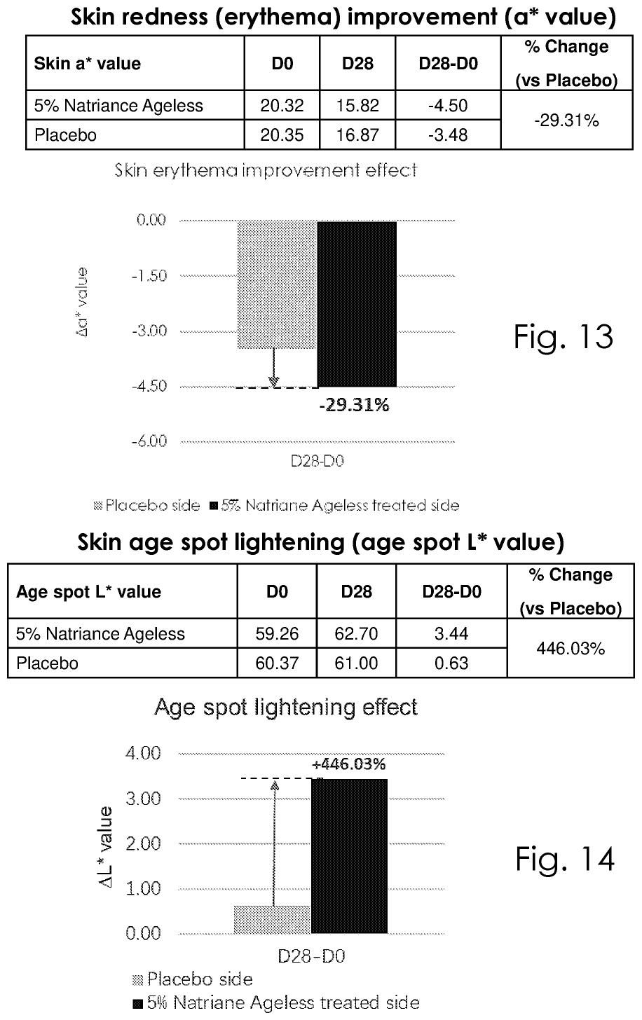

FIG. 13 shows the test result of human skin redness (a*) after the treatment with the composition according to the present invention and a placebo. Visia CR imaging of full face through image analysis showed that 5% Natriance Ageless (Active ingredient of example 1) can reduce skin redness (erythema) after 28 days treatment.

FIG. 14 shows the test result of human skin age spot (age spot L* value) after the treatment with the composition according to the present invention and a placebo. Visia CR imaging of full face through image analysis showed that 5% Natriance Ageless (Active ingredient of example 1) can lighten skin spots (as shown) after 28 days treatment.

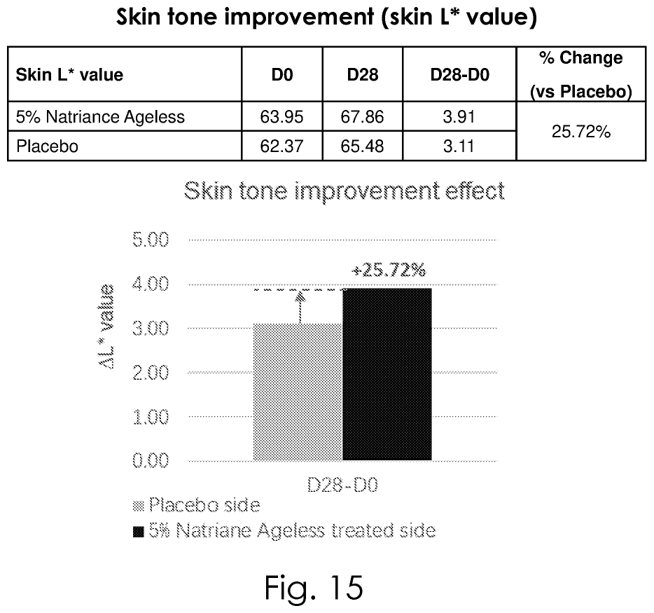

FIG. 15 shows the test result of human skin tone (skin L* value) after the treatment with the composition according to the present invention and a placebo. Visia CR imaging of full face through image analysis showed that 5% Natriance Ageless (Active ingredient of example 1) can lighten overall skin tone after 28 days treatment.

Conclusion:

Natriance Ageless (Active ingredient of example 1) can improve skin elasticity and firmness, reduce wrinkles such as crow's feet, refine skin pore, reduce skin erythema, lighten skin spots and improve skin tone.

What is claimed is:

1. A composition for improving the appearance of the skin comprising a physiologically acceptable medium and an effective amount of an active ingredient consisting of, based on the total weight of the active ingredient:
between 50% to 99.9%, preferably between 70% and 90% of water
between 5% to 20%, preferably between 10% and 15% of glycerin,
between 0.1% to 5.0%, preferably between 1% and 4% of Sodium PCA (Sodium Pyrrolidonecarboxylate),
between 0.1% to 3.0%, preferably between 0.5% and 2.0% of Erythritol,
between 0.1% to 2.0%, preferably between 0.3% and 1.0% of Carrageenan (Chondrus Crispus extract),
between 0.1% to 2.0%, preferably between 0.2% and 1.0% of Xanthan Gum,
between 0.1% to 3.0%, preferably between 0.5% and 2.0% of hydrolyzed Collagen,
between 0.001% to 0.02%, preferably between 0.002% and 0.01% of Hexapeptide-9,
optionally between 0.05% to 0.3%, preferably between 0.1% and 0.2% of Benzoic Acid,
optionally between 0.05% to 0.3%, preferably between 0.1% and 0.2% of Sorbic Acid.

2. The composition of claim 1 wherein the active ingredient consists of, based on the total weight of the active ingredient:
water 81.696%, glycerin 12%; sodium PCA 2.7%; erythritol 0.9%; carrageenan (Chondrus crispus extract) 0.6%; xanthan Gum 0.3%; benzoic Acid 0.18%; sorbic Acid 0.12%; hydrolyzed collagen 1.5% and Hexapeptide-9 0.004%.

3. The composition of claim 1, wherein the said active ingredient is at a concentration ranging from 1 to 10%.

4. The composition of claim 1, wherein the said active ingredient is at a concentration ranging from 1 to 5%.

5. The composition of claim 1, wherein the composition further comprises at least one additional active agent.

6. A cosmetic method for improving the appearance of the skin, said method comprising applying to the skin surface a composition according to claim 1, wherein the effects on the skin are selected among the skin signs of aging: improving skin elasticity, improving skin firmness, reducing wrinkles, lightening age spots.

7. The method of claim 6 wherein the said composition applied during at least 28 days.

8. A cosmetic method for improving the appearance of the skin, said method comprising applying to the skin surface a composition according to claim 1, wherein the effects on the skin include: refining skin pore, improving skin tone evenness and lightening the skin.

9. The method of claim 8 wherein the said composition is applied during at least 28 days.

10. A cosmetic method for improving skin moisturization, said method comprising applying to the skin surface a composition according to claim 1.

11. The method of claim 10 wherein the said composition is applied from 1 to 5 hours.

12. A cosmetic method for increasing the expression of collagen I, collagen III, collagen IV and collagen XVII in skin cells, comprising applying to the skin surface a composition according to claim 1, twice a day for 48 hours.
